(19) United States
(12) Patent Application Publication
(10) Pub. No.: US 2026/0248534 A1
(43) Pub. Date: Aug. 27, 2026

(54) System and method for aligning vertebrae in the correction of spinal deformities

(71) Applicant: Xiaoyu WANG, SAINT-BRUNO (CA)

(72) Inventor: Xiaoyu WANG, SAINT-BRUNO (CA)

(21) Appl. No.: 19/060,662

(22) Filed: Feb. 22, 2025

(51) Int. Cl. A61B 17/70; A61B 17/88

(52) U.S. Cl. CPC A61B 17/7004; A61B 17/701; A61B 17/705; A61B 17/7052; A61B 17/8869

(57) ABSTRACT

A system for aligning vertebrae to correct spinal deformities, comprising two transverse arms translating on two elongate bodies coupled by a gear pair mounted on a center body with revolute joints and ratchet units. The transverse arms and center body are connected to one side of the spine with three telescopic arms using three end-effectors. Auxiliary arms and end-effectors are available to connect the system to the other side of the spine. Coronal plane deformity is corrected by manipulating the angle between the elongate bodies with two manipulating handles to translate the apex of the spinal curve toward the midline of the patient; adjusting the lengths of the telescopic and auxiliary arms to correct the sagittal and transverse plane deformities. The end-effector positions are locked by engaging the ratchet units to keep the achieved corrections while the spinal rods are fixed on the spine through bone-implants.

### CROSS-REFERENCE TO RELATED APPLICATIONS

None.

### BACKGROUND OF THE INVENTION

The present invention relates to apparatus and method for correction of spinal deformities, such as scoliosis and hyperkyphosis.

Posterior spinal instrumentation is a main treatment option for severe spinal deformity manifested with an abnormal spinal curve. It uses two spinal rods precontoured to have the same shape as a normal spinal curve. The rods are attached to the left and right side of the spine, respectively, using bone implants. The bone implants generally feature a saddle-like head and a set screw. The rod can be connected to the implant and free to rotate around and translate along its lengthwise axis. The rod can be fixed on the implant by tightening the set screw. Initial curve correction is achieved as the rods are connected to but not fixed on the implants. Further correction is achieved by manipulating relative position and orientation between the spine, the rods, and the implants. The instrumentation is completed by fixing the spinal rods to all implants.

In the course of connecting the rod to implants and during the corrective manipulations, mechanical stresses in the rod evolve temporally and spatially. A major challenge not yet successfully addressed in the prior arts is that the rod can deform far beyond its elastic limit due to transient overload, especially when the spinal curve is very stiff, resulting in loss of intraoperative correction, compromised spinal rod fatigue strength, and even an inability to achieve adequate deformity correction.

There is a need for a solution to reduce the risk of material yielding of the spinal rod in the course of spinal deformity correction.

### SUMMARY OF THE INVENTION

This Summary is provided to explain the essential concepts of the present invention; details are provided in the Detailed Description. It is not intended to identify key features or essential features of the claims, nor is it intended to limit the scope of the claims.

The present invention seeks to provide a solution to reduce the risk of material yielding of the spinal rod in the course of spinal deformity correction. The underlying principle is that the spinal rod will not be used until the abnormal spinal curve has been corrected and maintained by a system, and there has been substantial post-correction spinal stress relaxation.

In one embodiment of applicant's present invention, a system is provided for aligning vertebrae to correct an abnormal spinal curve, maintaining intraoperative correction as spinal stresses relax, and bearing reaction forces of the spine as a spinal rod is connected to and fixed on bone implants.

The system comprises a primary motion unit, three telescopic arms, and three end-effectors. The primary motion unit comprises a center body, two elongate bodies, and two transverse arms. The two elongate bodies are respectively connected on the center body with a revolute joint and coupled by a gear pair of a gear ratio of one to constrain their rotations with respect to the center body to be equal and in the opposite directions. Each elongate body is also connected to the center body with a ratchet unit which has an operating handle to be used to engage or disengage the ratchet. The elongate bodies are free to rotate with respect to the center body when both ratchet units are disengaged. When engaged, one ratchet prevents the rotation of the elongate bodies about the center body in one direction; the other ratchet prevents the rotation of the elongate bodies about the center body in the other direction. The elongate bodies each feature a manipulating handle. The primary spinal deformity connection motion is generated in the coronal plane by pushing the two manipulating handles toward each other or pulling them away from each other to change the angle between the two elongate bodies. The two transverse arms are respectively mounted on the two elongate bodies with a prismatic or helical joint enabling the system to be adjusted to the size of the individual spinal curve.

The center body and the two transverse arms are respectively connected to the three telescopic arms which are connected to one side of the spinal column at three locations using three end-effectors, respectively. The motion generated by the primary motion unit corrects the coronal spinal deformity by translating the apex of the coronal spinal curve toward the midline of the patient. The lengths of the telescopic arms are respectively adjusted with three controlling screws to correct the spinal deformity in the sagittal plane.

The system includes one to three auxiliary arms and additional end-effectors which are connected to the other side of the spinal column in the same manner as the primary telescopic arms. The auxiliary arms are respectively connected to the primary telescopic arms with an interconnector. The auxiliary arms are also telescopic and their lengths are respectively adjusted with adjusting screws. Differential adjusting of the lengths of the auxiliary arms with respect to the lengths of the primary telescopic arms corrects the spinal deformity in the transverse plane.

Once the desired correction is achieved, the two ratchet units are engaged to lock the positions of the end-effectors so as to keep the achieved correction for an appropriate time to allow substantial spinal stress relaxation to occur. In the meantime, the spinal rods are contoured to the corrected spinal shape and inserted and locked onto the bone-implants while the system bears all the reaction forces from the spine. The system is disconnected from the spine after the spinal rods are locked onto all the bone-implants and significant spinal stress relaxation is believed to has occurred.

### BRIEF DESCRIPTION OF DRAWINGS

FIG. 1—Perspective view of the assembly of the system for aligning vertebrae in the correction of spinal deformities.

FIG. 2—Major components of the system for aligning vertebrae in the correction of spinal deformities.

FIG. 3—Key components of the system for aligning vertebrae in the correction of spinal deformities.

FIG. 4—Schematic perspective view of the kinematic joint groups associated with the key components of the system for aligning vertebrae in the correction of spinal deformities.

FIG. 5—Illustration of relative displacements between components of the system for aligning vertebrae in the correction of spinal deformities.

FIG. 6—Exploded perspective view of the assembly of the center body and the two elongate bodies.

FIG. 7—Top view of the assembly of the center body and the two elongate bodies.

FIG. 8—Perspective view of the assembly of the transverse arm.

FIG. 9—Perspective view of the assembly of the telescopic arm.

FIG. 10—Perspective view of the assembly of the auxiliary arm.

FIG. 11—Perspective view of the assembly of the rod gripper with the locking jaw closed.

FIG. 12—Perspective view of the assembly of the rod gripper with the locking jaw opened.

FIG. 13—Illustration of the rod gripper operation.

FIG. 14—Perspective view of the assembly of bone implant, spinal rod, rod-implant connector, and rod.

FIG. 15—Perspective view of the assembly of bone-implant and rod-implant connector.

FIG. 16—Exploded perspective view of the assembly of bone-implant and rod-implant connector.

FIG. 17—Perspective and orthographic views of the implant-receiver member of the rod-implant connector.

FIG. 18—Cross section view of the assembly of the rod-implant connector and bone implant.

FIG. 19—Perspective and orthographic views of the locking body of the rod-implant connector.

FIG. 20—Perspective view of the assembly of the implant receiver member and the locking bodies of the rod-implant connector.

FIG. 21—Perspective and orthographic views of the rod receiver member of the rod-implant connector.

FIG. 22—Perspective view of the assembly of the implant receiver member, the locking bodies, and the rod receiver member of the rod-implant connector.

FIG. 23—Perspective view of the assembly of the rod-implant connector.

FIG. 24—a) Deformed spinal segment, b) Bone-implant are implanted, c) Rod-implant connectors are connected to bone-implants, d) rods are connected to the rod-implant connectors.

FIG. 25—Sagittal and transverse plane corrections are done by adjusting the lengths of the telescopic arms and the auxiliary arms.

FIG. 26—Coronal plane correction is done by manipulating the angle between the two elongate bodies using the two manipulating handles

### DETAILED DESCRIPTION OF THE INVENTION

Detailed description of the Invention is provided through the presentation of an embodiment of the present invention. The embodiment is chosen in order to best explain the working principles of the invention and its applications and practical uses. The embodiment is not to limit the invention to the particular form disclosed.

In the following, purely for convenience, certain terminology is used and is not to be taken as a limitation on the invention. The terminology includes the words and terms specifically mentioned, derivatives thereof, and words and terms of similar import.

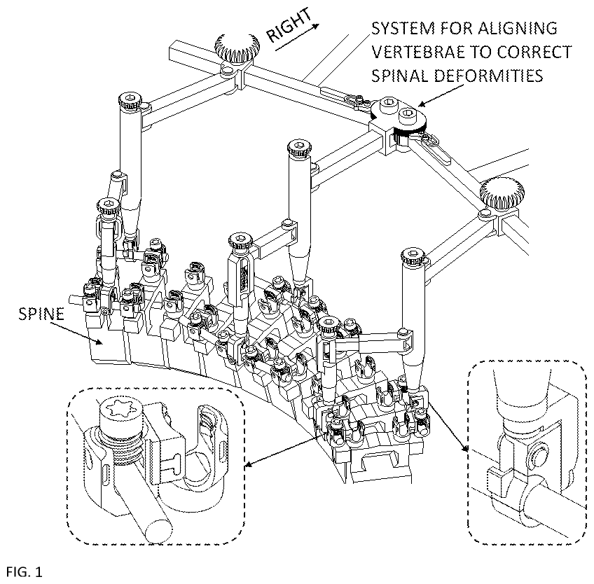

With reference to FIG. 1, the system is connected to one side or two sides of the spinal column.

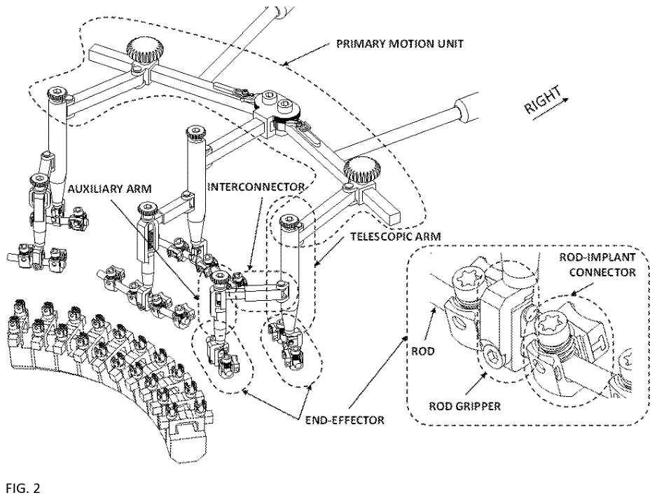

With reference to FIG. 2, the system comprises a primary motion unit, three telescopic arms, three end-effectors, and additional auxiliary arms, interconnectors, and end-effectors.

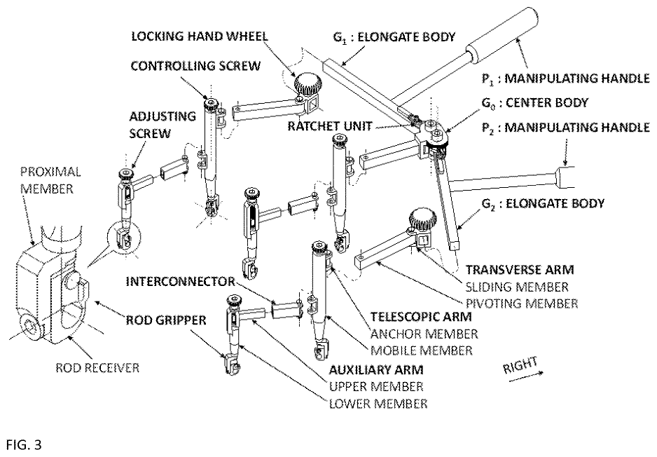

With reference to FIG. 3, the primary motion unit comprises a center body, two elongate bodies each featuring a manipulating handle, two ratchet units, and two transverse arms. Each transverse arm comprises a sliding member, a pivoting member, and a locking screw with a locking hand wheel. Each telescopic arm comprises an anchor member and a mobile member as well as a controlling screw featuring a hand wheel. The auxiliary arm comprises an upper member, a lower member, and an adjusting screw with a hand wheel. The rod gripper comprises a proximal member and a receiving jaw.

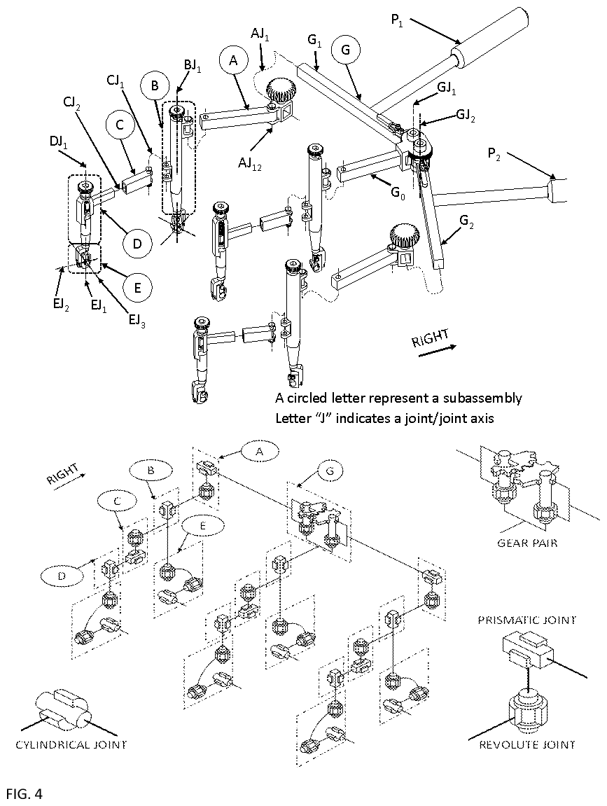

With reference to FIG. 4, the kinematic joint group (G) corresponds to connections between the two elongate bodies and the center body, comprising two revolute joints couple by a gear pair. Group (A) corresponds to connections to the transverse arm, comprising a prismatic joint and a revolute joint where the prismatic joint can be replaced with a helical joint. Group (B) corresponds to the telescopic arm having a single prismatic joint which can be replaced with a helical joint. Group (C) corresponds to the interconnector, comprising a revolute joint and a prismatic joint. Group (D) corresponds to the auxiliary arm, having a single prismatic joint which can be replaced with a helical joint. Group (E) corresponds to the rod gripper, comprising two revolute joints as well as a cylindrical joint between the rod gripper and the rod.

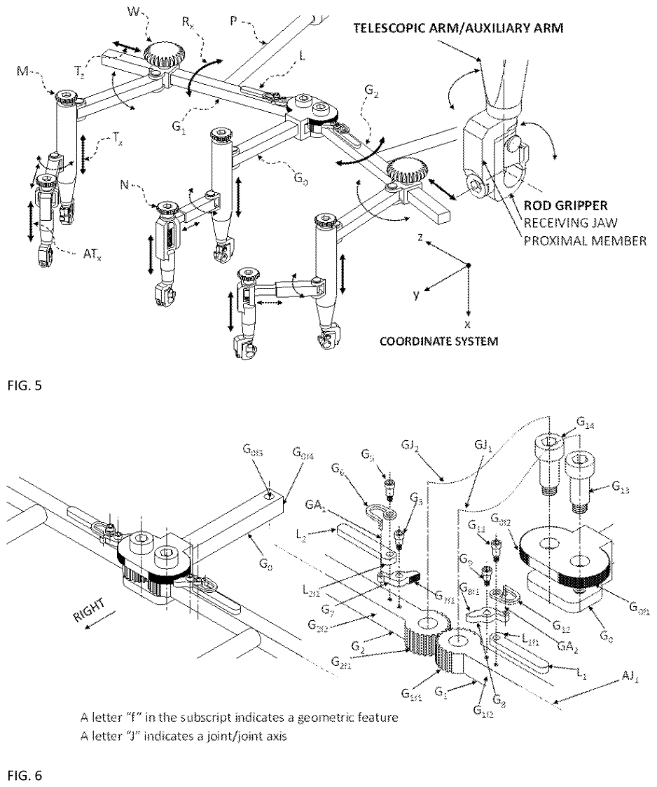

With reference to FIG. 4 and FIG. 5, the kinematic joint group (G) enables a relative rotation Rₓ of the two elongate bodies in the yz-plane of the coordinate system. The group (A) enables a translation T_{z} of the transverse arm along the elongate body which can be locked with the locking hand wheel W, and a rotation of the transverse arm in the yz-plane. The group (B) enables a translation Tₓ of the mobile member of the telescopic arm along the x-axis which is controlled with the hand wheel M. The group (C) enables a rotation and a translation of the auxiliary arm with respect the telescopic arm in the yz-plane. The group (D) enables a translation ATₓ of the lower member of the auxiliary arm relative to its upper member along the x-axis which is controlled with the hand wheel N. The group (E) enables a 2-degree-of-freedom rotation of the receiving jaw of the rod gripper with respect to the telescopic or auxiliary arm.

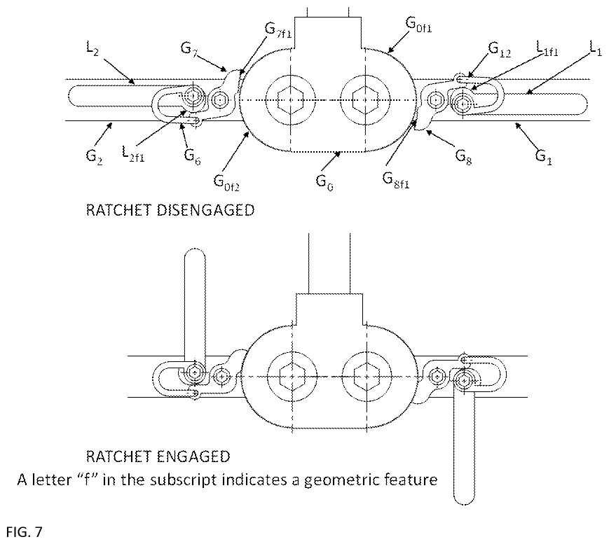

With reference to FIG. 6 and FIG. 7, the elongate bodies G₁ and G₂ are connected to the center body G₀ with two revolute joints through pins G₁₃ and G₁₄ whose axes of rotation are GJ₁ and GJ₂, respectively. The elongate bodies G₁ and G₂ are coupled by sector gears G_{1f1} and G_{2f1} with GJ₁ and GJ₂ as their axes. The elongate bodies G₁ and G₂ are also each connected to the center body G₀ with a ratchet unit. The ratchet pawls G₈ and G₇ are pivotally mounted on the elongate bodies G₁ and G₂ with pins G₉ and G₃, respectively. The camming levers L₁ and L₂ are pivotally mounted on the elongate bodies G₁ and G₂ with pins G₁₁ and G₅, respectively. The U-shaped springs G₁₂ and G₆ are pivotally mounted on the elongate bodies G₁ and G₂ with pins G₁₁ and G₅, respectively. The U-shaped springs G₁₂ and G₆ are pivotally engaged with the ratchet pawls G₈ and G₇ around the axes GA₂ and GA₁, respectively. Pressures exerted by the U-shaped springs G₁₂ and G₆ keep the ratchet pawls G₈ and G₇ in contact with the camming surfaces L_{1f1} and L_{2f1} on levers L₁ and L₂. The ratchet pawls G₈ and G₇ features multiple teeth G_{8f1} and G_{7f1} which are shaped and sized to match the ratchet gear teeth G_{0f1} and G_{0f2} on the center body G₀. When the camming levers L₁ and L₂ are turned to be aligned with the elongate bodies G₁ and G₂, the teeth G_{8f1} and G_{7f1} on the ratchet pawls G₈ and G₇ are cammed away from and disengaged with the teeth G_{0f1} and G_{0f2} on the center body G₀; the angle between the elongate bodies G₁ and G₂ can be manipulated by pulling away from each other or pushing toward each other the two manipulating handles P₁ and P₂ on the elongate bodies (FIG. 3). When one of the camming levers L₁ or L₂ is turned clockwise to be perpendicular to its corresponding elongate body, the teeth on the corresponding ratchet pawl is engaged with the ratchet gear teeth on the center body G₀ under the pressure from the U-shaped spring; the elongate body can only be turned around the center body G₀ in one direction. In the same manner, rotation of the elongate body around the center body G₀ in the other direction is blocked when the other lever is turned to be perpendicular to its corresponding elongate body.

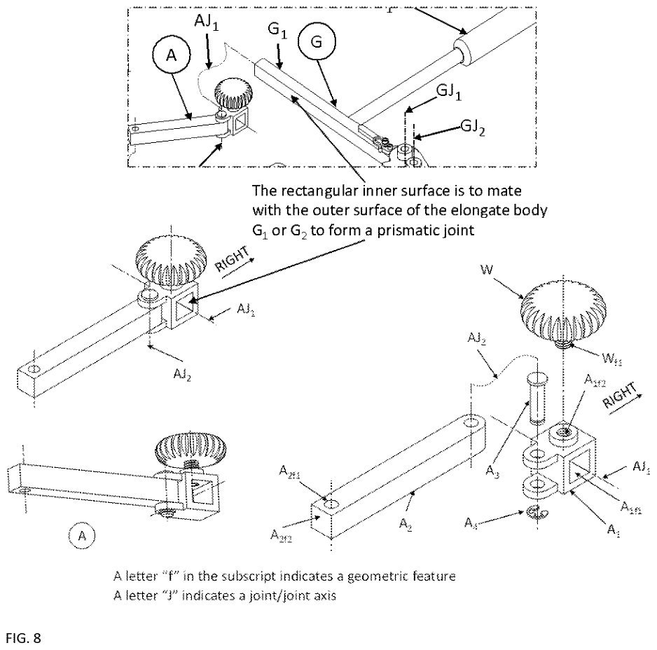

With reference to FIG. 8, the sliding member A₁ of the transverse arm is connected to the pivoting member A₂ of the transverse arm with a revolute joint around the axis AJ₂ through pin A₃ and retaining ring A₄. The sliding member A₁ of the transverse arm is connected to the elongate body G₁ with a prismatic joint along the axis AJ₁ through a clearance fit between the inner surface A_{1f1} of the sliding member A₁ and the outer surface G_{1f2} of the elongate body G₁ (FIG. 6). It is understood that the prismatic joint can be replace with a helical joint to fulfil the same function. The locking hand wheel W features a threaded cylindrical protrusion W_{f1} which is in a clearance fit with the threaded hole A_{1f2} of the sliding member A₁. Turning clockwise the locking hand wheel W locks the sliding member of the transverse arm on the elongate body G₁.

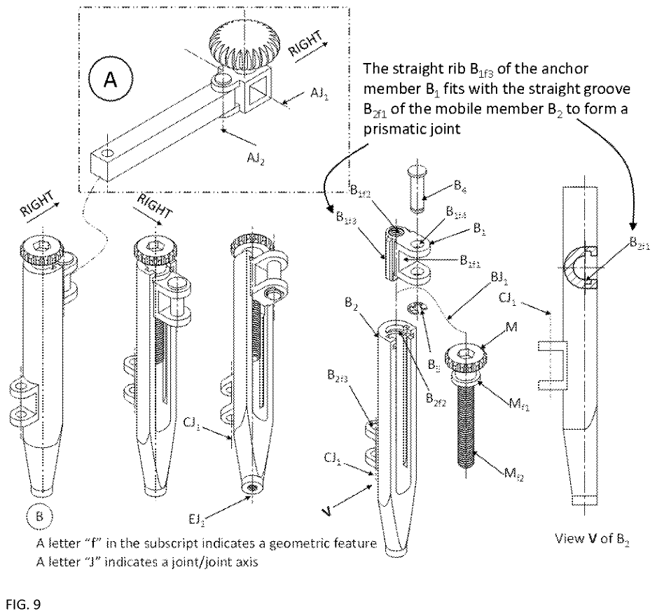

With reference to FIG. 9, the anchor member B₁ of the telescopic arm is connected to the mobile member B₂ of the telescopic arm with a prismatic joint along the axes BJ₁ through a clearance fit between the straight rib B_{1f3} of the anchor member B₁ and the straight groove B_{2f1} of the mobile member B₂. It is understood that the prismatic joint can be replaced with a helical joint to fulfil the same function. The controlling screw M features a circular rib M_{f1} which is in a clearance fit with a circular groove B_{2f2} in the mobile member B₂, forming a revolute joint. The threaded portion of the controlling screw W is in a clearance fit with the threaded hole B_{1f2} of the anchor member B₁. Turning the hand wheel on the controlling screw M translates the mobile member B₂ with respect to the anchor member B₁ along the axis BJ₁. The anchor member B₁ of the telescopic arm is connected to the pivoting member A₂ of the transverse arm with a fixed joint through the fit of the pin B₄ with the hole B_{1f4} of B₁, hole A_{2f1} of A₂ (FIG. 8), retaining ring B₅, and the contact between the flat surface B_{1f1} of B₁ and the flat surface A_{2f2} of A₂ (FIG. 8). The anchor member B₁ of the telescopic arm is connected to the center body G₀ of the primary motion unit (FIG. 6) with a fixed joint through the fit of the pin B₄ with the hole B_{1f4} of B₁, hole G_{0f3} of G₀ (FIG. 6), retaining ring B₅, and the contact between the flat surface of B_{1f1} of B₁ and the flat surface G_{0f4} of G₀ (FIG. 6).

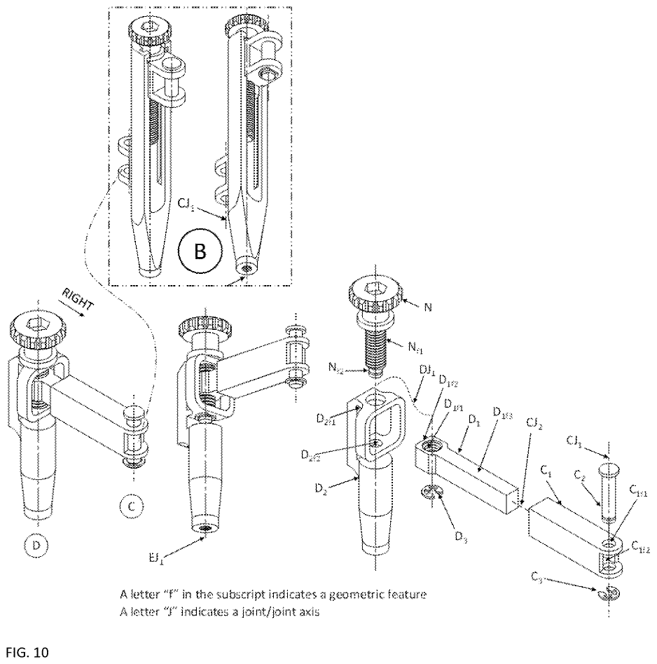

With reference to FIG. 10, the interconnector C₁ is connected to the mobile member B₂ of the telescopic arm (FIG. 9) with a revolute joint around the axis CJ₁ through the fit of the pin C₂ with the hole C_{1f1} of C₁, hole B_{2f3} of B₂ (FIG. 9), and the retaining ring C₃. The interconnector C₁ is connected to the upper member D₁ of the auxiliary arm with a prismatic joint along the axis CJ₂ through a clearance fit between the inner surface C_{1f2} of C₁ and the outer surface D_{1f3} of D₁. The upper member D₁ of the auxiliary arm is connected to the lower arm D₂ of the auxiliary arm with a prismatic joint along the axis DJ₁ through the contact between the flat surface D_{1f2} of D₁ and the flat surface D_{2f1} of D₂, and the fit of the adjusting screw N with the threaded hole D_{1f1} of D₁, the hole D_{2f2} of D₂, and the retaining ring D₃; these fits also form a helical joint between the adjusting screw N and the upper member D₁. It is understood that this prismatic joint can be replaced with a helical joint to fulfil the same function. Turning the hand wheel on the adjusting screw N translates the lower member D₂ with respect to the upper member D₁ along the axis DJ₁.

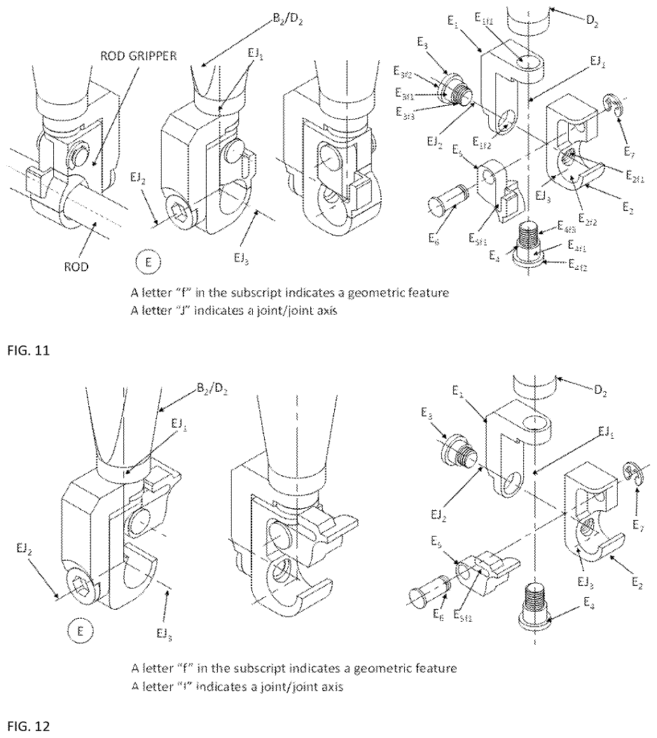

With reference to FIG. 11 and FIG. 12, the rod gripper assembly comprises a proximal member E₁, a receiving jaw E₂, screw pins E₃ and E₄, and locking jaw E₅. Each screw pin has a smooth pin portion, a stopping shoulder at one end of the smooth pin portion and a threaded portion at the other end of the smooth pin portion. The threaded portion E_{4f3} of the screw pin E₄ goes through the hole E_{1f1} of the proximal member E₁ and is threaded and tight-fit into a tapped hole of the telescopic or auxiliary arm, resulting in a revolute joint around the axis EJ₁ between the proximal member E₁ and the telescopic or auxiliary arm. The threaded portion E_{3f3} of the screw pin E₃ goes through the hole E_{1f2} of the proximal member E₁ and is threaded and tight-fit into the tapped hole E_{2f1} of the receiving jaw E₂, resulting in a revolute joint around the axis EJ₂ between the proximal member E₁ and the receiving jaw E₂. The receiving jaw E₂ has a hook-like shape with a partial cylindrical inner surface E_{2f2} to receive a rod. The locking jaw E₅ is mounted on the receiving jaw E₂ with a revolute joint using the pin E₆ and retaining ring E₇.

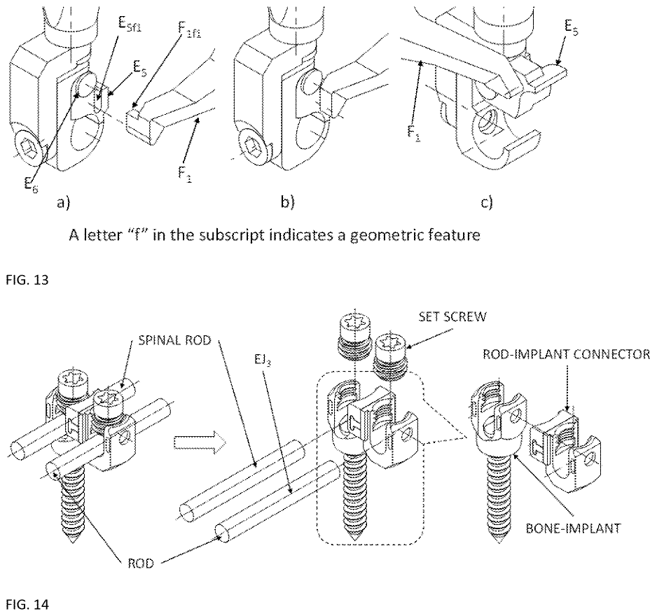

With reference to FIG. 13, the opening and closing of the locking jaw E₅ is operated using the jaw-driver F₁. The locking jaw E₅ features a recess E_{5f1} and the jaw-driver F₁ features a protrusion F_{1f1} that is shaped, sized and disposed to fit into the recess E_{5f1}. Turning the jaw-driver F₁ counterclockwise around the pin E₆ lifts up and opens the locking Jaw E₅ to allow a rod to be seated in and mated with the inner cylindrical portion E_{2f2} of the receiving jaw E₂ (FIG. 11) . Turning the jaw-driver F₁ clockwise around the pin E₆ pushes down and closes the locking Jaw E₅ to prevent the rod from disengaging from the receiving jaw.

With reference to FIG. 14, the rod is connected to the spine through rod-implant connectors and commonly used bone-implants, which feature an implant head having a center pocket on one side and two lateral recesses respectively on two other sides adjacent to the center pocket side.

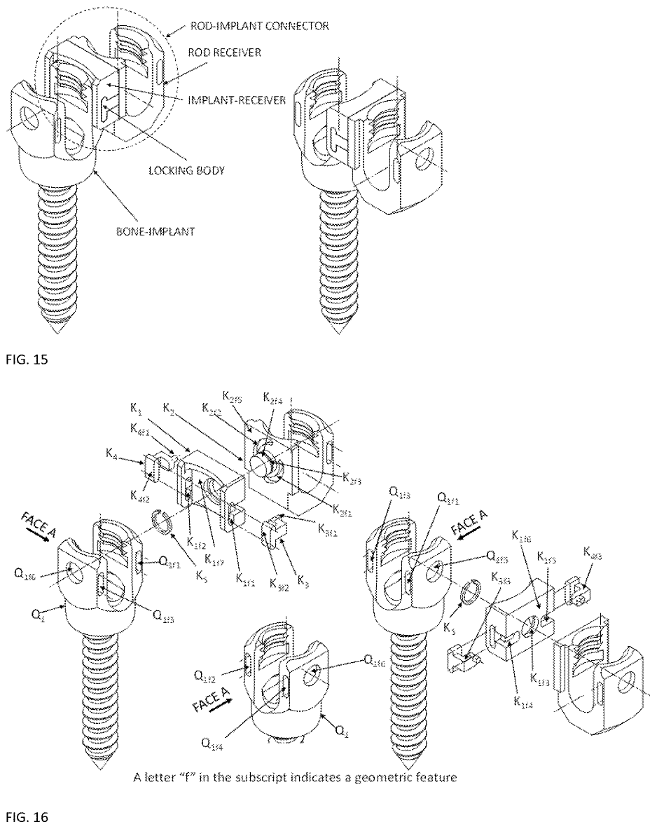

With reference to FIG. 15 and FIG. 16, the rod-implant connector comprises an implant receiver member, a rod receiver member, and two locking bodies. When assembled together with the bone-implant, the implant receiver member is sandwiched between the bone-implant and the rod-receiver member. The head Q₁ of a commonly used bone-implant features a center pocket Q_{1f5} (a center pocket Q_{1f6} on the opposite side) and two lateral recesses Q_{1f1} and Q_{1f2}. The surface K_{1f7} of the implant receiver member K₁ is shaped and sized to mate with the bone-implant on the side of the center packet Q_{1f5} and the sides of the two lateral recesses Q_{1f1} and Q_{1f2} of the bone-implant Q₁. The surface K_{1f6} of the implant receiver member K₁ is a flat surface, which is in contact with the flat surface K_{2f5} on the rod receiver member K₂.

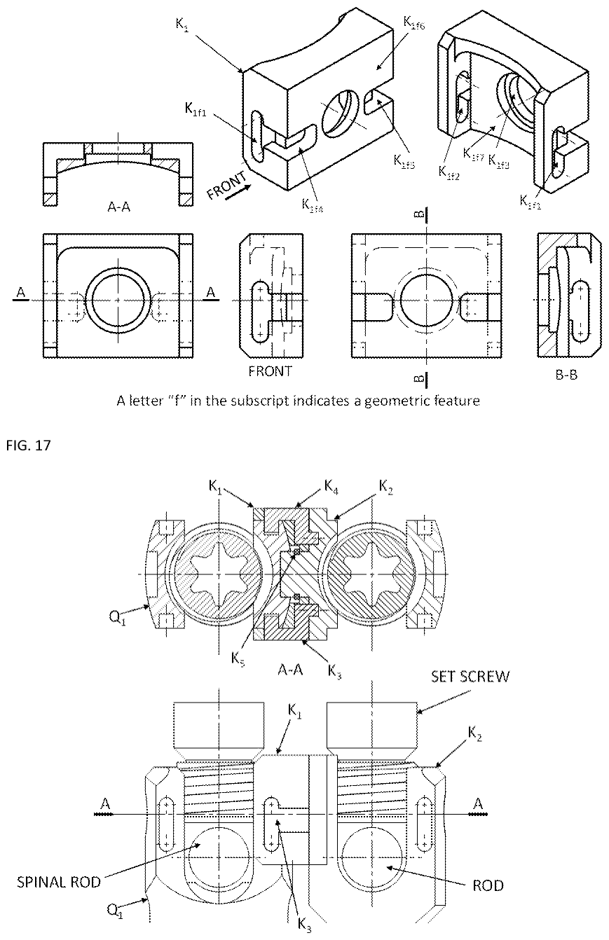

The implant receiver member K₁ has a center opening K_{1f3} and the rod receiver member K₂ has a center cylindrical protrusion K_{2f4} with a circular groove K_{2f3} in the midway; they are shaped, sized, and disposed such that the protrusion K_{2f4} mates with both the center opening K_{1f3} of the implant receiver K₁ and the center pocket Q_{1f5} of the bone-implant Q₁, and the retaining ring K₅ is mounted into the circular groove K_{2f3} to form a revolute joint between the implant receiver member K₁ and the rod receiver member K₂ (FIG. 17 and FIG. 18).

With reference to FIG. 16 and FIG. 17, the implant receiver member K₁ has two lateral openings K_{1f1} and K_{1f2} sized, shaped, and disposed to be aligned respectively with the two lateral recesses Q_{1f1} and Q_{1f2} of the bone-implant Q₁ enabling the implant receiver member K₁ to be latched and thus fastened onto the bone-implant Q₁.

With reference to FIG. 17, on the flat surface K_{1f6} of the implant receiver member K₁, two straight grooves K_{1f4} and K_{1f5} are made in two radial directions of the center opening K_{1f3}.

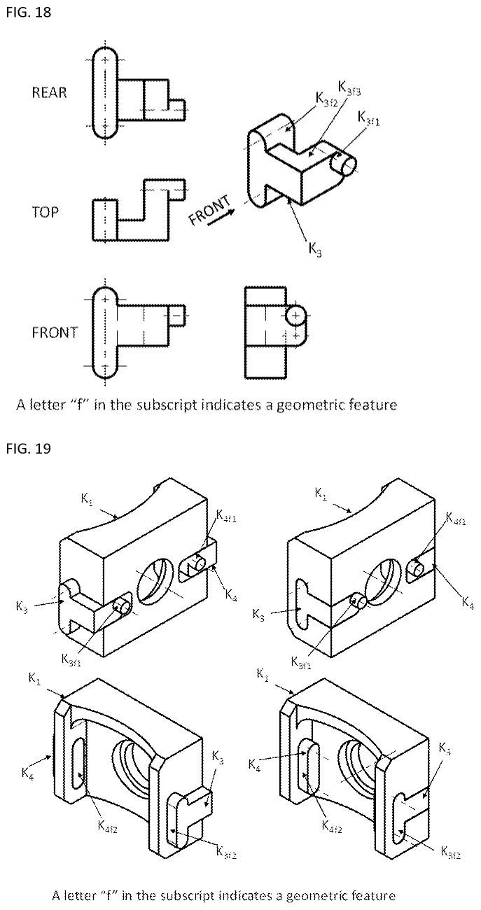

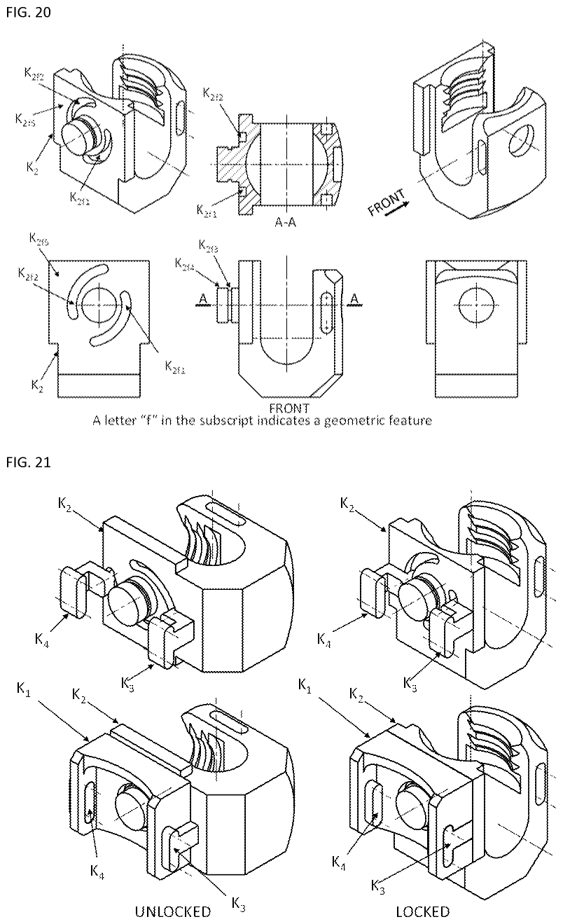

With reference to FIG. 19, the portion K_{3f3} of the locking body K₃ is shaped and sized such that said portion mates with and slides in the straight groove K_{1f4} of the implant receiver K₁ (FIG. 17). The portion K_{3f2} of the locking body K₃ is shaped and sized such that this portion slides into and mates with both the lateral opening K_{1f1} of the implant receiver member K₁ (FIG. 17 and FIG. 20) and the lateral recess Q_{1f1} of the bone-implant Q₁ to latch and thus lock the implant-receiver member K₁ onto the bone-implant Q₁ (FIG. 16 and FIG. 18).

With reference to FIG. 21, on the flat surface K_{2f5} of the rod receiver member K₂, two cam grooves K_{2f1} and K_{2f2} are made.

With reference to FIG. 19, the locking body K₃ features a protrusion K_{3f1} shaped, sized, and disposed to fit into the cam groove K_{2f1} of K₂ (FIG. 21).

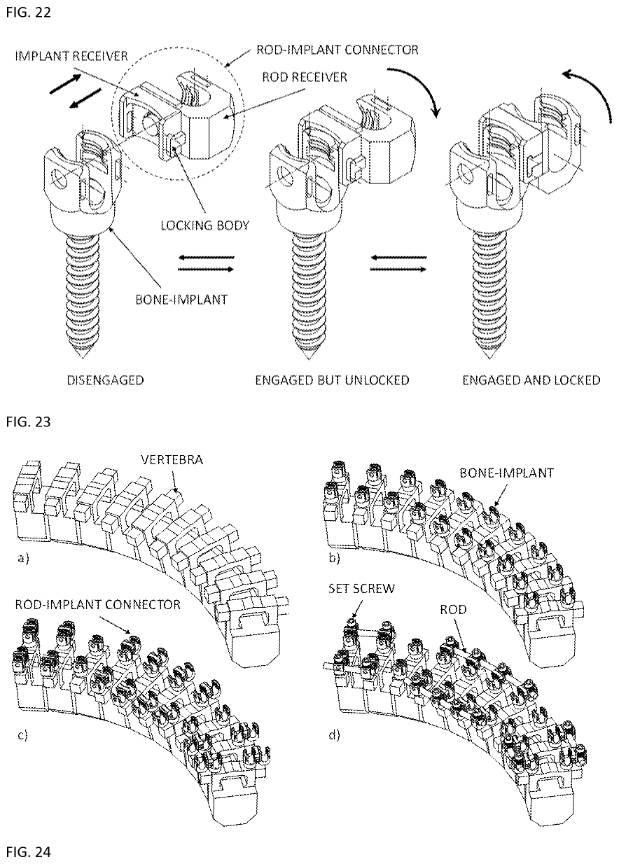

With reference to FIG. 22, turning the rod receiver member K₂ with respect to the implant receiver member K₁ clockwise cams the locking bodies K₃ and K₄ away and disengages them from the lateral recesses of the bone-implant enabling the implant receiver K₁ to be separated from the bone-implant; turning the rod receiver member K₂ with respect to the implant receiver member K₁ counterclockwise cams the locking bodies K₃ and K₄ into the lateral recesses of the bone-implant, latches and locks the implant receiver member onto the bone implant (FIG. 23).

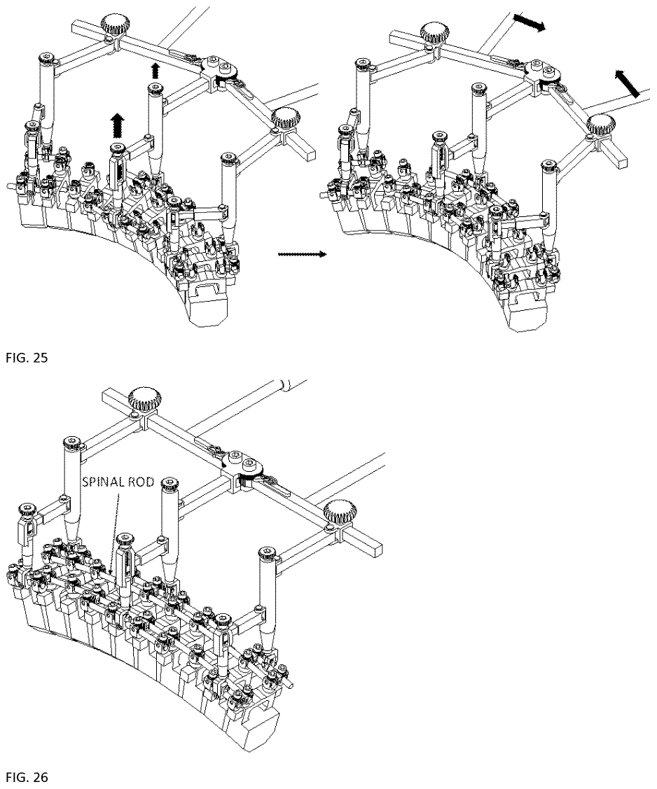

After the bone-implants are implanted at the proximal, apical, and distal vertebral levels of the spinal curve, the rod-implant connectors are connected to the bone-implants and the rods are connected to the rod-implant connectors (FIG. 24). The ratchet units are disengaged and the positions of the two transverse arms and the angle between the two elongate bodies are adjusted to the spinal curve to connect the system to the spinal column (FIG. 25). Sagittal and transverse plane corrections are done by adjusting the lengths of the telescopic and auxiliary arms (FIG. 25). Coronal plane correction is done by manipulating the angle between the two elongate bodies using the two manipulation handles (FIG. 26). Once the desired corrections are achieved, the ratchet units are engaged to maintain the corrections. Spinal rods are then contoured to match the alignment of the bone-implants, inserted, and locked on the bone-implants to secure the corrections and allow the system to be disconnected. The system is disconnected from the spinal column when significant spinal stress relaxation is believed to have occurred to maximally reduce the risk of material yielding of the spinal rods.

1. A system for aligning vertebrae to correct spinal deformities, maintaining intraoperative correction, and bearing reaction forces of the spine comprising: a primary motion unit and three telescopic arms; the primary motion unit comprises a center body, two elongate bodies, and two transverse arms; the two elongate bodies are connected respectively to the center body with a revolute joint and extend in opposite directions; the two elongate bodies are coupled by a gear pair with a gear ratio of one to constrain the rotations of the elongate bodies with respect to the center body to be equal and in the opposite directions; the transverse arms each comprise a sliding member and a pivoting member, the pivoting member being connected to the sliding member with a revolute joint; the sliding members of the transverse arms are respectively connected to the elongate bodies with a prismatic or helical joint whose translation can be locked with a locking screw featuring a locking hand wheel; the pivoting members of the transverse arms and the center body of the primary motion unit are respectively connected to the three telescopic arms with a fixed joint.

2. The system of claim 1, wherein the elongate bodies are respectively connected to the center body with a ratchet unit which has an operating handle to be used to engage or disengage the ratchet; the elongate bodies are free to rotate with respect to the center body when both ratchet units are disengaged; when engaged, one ratchet prevents the rotation of the elongate bodies with respect to the center body in one direction; the other ratchet prevents the rotation of the elongate bodies with respect to the center body in the other direction.

3. The system of claim 1, wherein the telescopic arms each comprise: an anchor member and a mobile member connected with a prismatic or helical joint whose translation is controlled with a controlling screw featuring a hand wheel; the anchor member is connected to the pivoting member of the transverse arm or the center body of the primary motion unit with a fixed joint.

4. The system of claim 1 includes three end-effectors respectively connected to the mobile members of the telescopic arms with a revolute joint; the three end-effectors are connected to the spinal column through bone-implants.

5. The end-effector of claim 4 includes a rod gripper, a rod, and a rod-implant connector.

6. A rod gripper assembly for holding a rod and transmitting motion and force to the rod, comprising: a proximal member, two screw pins, a receiving jaw, and a locking jaw; each screw pin has a smooth pin portion, a stopping shoulder at one end of the smooth pin portion and a threaded portion at the other end of the smooth pin portion; the threaded portion of the first screw pin goes through a hole of the proximal member and is threaded and tight-fit into a tapped hole of a mechanical component, resulting in a revolute joint between the proximal member and the mechanical component; the threaded portion of the second screw pin goes through another hole of the proximal member and is threaded and tight-fit into a tapped hole of the receiving jaw, resulting in a revolute joint between the proximal member and the receiving jaw; the receiving jaw has a hook-like shape with a partial cylindrical inner surface to receive a rod; the locking jaw is mounted on the receiving jaw with a revolute joint; using a jaw-driver, the locking jaw is lifted up to allow the rod to be engaged with the receiving jaw from the opening side of the hook-like shape receiving jaw and is closed down with the jaw-driver to prevent the rod from disengaging from the receiving jaw.

7. The system of claim 1 includes one to three auxiliary arms each comprising: an interconnector, an upper member, and a lower member; the upper member and lower member are connected with a prismatic or helical joint whose translation is controlled with an adjusting screw featuring a hand wheel; the upper member is connected to the interconnector with a prismatic joint; the interconnector is connected to the mobile member of the telescopic arm with a revolute joint; a revolute joint is used to connect the lower member of the auxiliary arm to the proximal member of a rod gripper.

8. A rod-implant connector assembly for connecting a rod onto a commonly used bone-implant which features a center pocket on one side and two lateral recesses respectively on two other sides adjacent to the center pocket side, comprising: an implant receiver member, a rod receiver member, and one or two locking bodies; when assembled together with the bone-implant, the implant receiver member is sandwiched between the bone-implant and the rod-receiver member; one side of the implant receiver member is shaped and sized to mate with the bone-implant on the side of the center packet and the sides of the two lateral recesses of the bone-implant; the opposite side of the implant receiver member has a flat surface which is in contact with a flat surface on the rod receiver member; the implant receiver member has a center opening and the rod receiver member has a center cylindrical protrusion with a circular groove in the midway; they are shaped, sized, and disposed such that the protrusion mates with both the center opening of the implant receiver member and the center pocket of the bone-implant, and a retaining ring is mounted into the circular groove to form a revolute joint between the implant receiver member and the rod receiver member; the implant receiver member has two lateral openings sized, shaped, and disposed to be aligned respectively with the two lateral recesses of the bone-implant enabling the implant receiver member to be latched and thus fastened onto the bone-implant; on the flat surface of the implant receiver member in contact with the flat surface of the rod receiver member, two straight grooves are made in two radial directions of the center opening; a portion of each locking body is shaped and sized such that said portion mates with and slides in the straight groove; the portion of each locking body outside the straight groove is shaped, sized, and disposed such that this portion slides into and mates with both the lateral opening of the implant receiver member and the lateral recess of the bone-implant to latch and thus lock the implant-receiver member onto the bone-implant; on the flat surface of the rod receiver member, two cam grooves are made; on each locking body, a protrusion is made to fit into one of the cam grooves; turning the rod receiver member with respect to the implant receiver member clockwise cams the locking bodies away and disengages them from the lateral recesses of the bone-implant enabling the implant receiver to be separated from the bone-implant; turning the rod receiver member with respect to the implant receiver member counterclockwise cams the locking bodies into the lateral recesses of the bone-implant, latches and locks the implant receiver member onto the bone implant.
